(19) United States
(12) Patent Application Publication
(10) Pub. No.: US 2006/0084878 A1
(43) Pub. Date: Apr. 20, 2006

(54) PERSONAL COMPUTER-BASED VITAL SIGNS MONITOR

(75) Inventors: Matthew John Banet, Del Mar, CA (US); Robert Murad, San Diego, CA (US); Bruce Driver, San Diego, CA (US); Manuel Eduardo Jaime, Solana Beach, CA (US); Henk Visser, San Diego, CA (US); Brett George Morris, San Diego, CA (US)

Correspondence Address: Triage Wireless, Inc.;Matthew John Banet, 6540 LUSK BLVD., C200, SAN DIEGO, CA, 92121, US

(73) Assignee: TRIAGE WIRELESS, INC., San Diego, CA (US)

(21) Appl. No.: 10/906,342

(22) Filed: Feb. 15, 2005

Related U.S. Application Data
(63) Continuation of application No. 10/967,610, filed on Oct. 18, 2004, now Pat. No. 7,004,907.

(51) Int. Cl. A61B 5/02

(52) U.S. Cl. 600/485; 128/920

(57) ABSTRACT

The invention provides a system for measuring blood pressure from a patient that includes: 1) an optical module featuring systems for measuring signals from the patient, serial communication, and power management; 2) an external computing device configured to attach to the optical module, supply power to the optical module, and receive information from the optical module through the system for serial communication; and 3) an algorithm, operating on the external computing device, that processes information received through the system for serial communication to determine the patient's blood pressure.

This application is a continuation-in-part application of U.S. patent application Ser. No. 10/967,610, filed Oct. 18, 2004.

### STATEMENT REGARDING FEDERALLY SPONSORED RESEARCH OR DEVELOPMENT

Not Applicable

### BACKGROUND OF THE INVENTION

1. Field of the Invention

The present invention relates to medical devices for monitoring vital signs such as heart rate, pulse oximetry, and blood pressure.

2. Description of the Related Art

Pulse oximeters are medical devices featuring an optical module, typically worn on a patient's finger or ear lobe, and a processing module that analyzes data generated by the optical module. The optical module typically includes first and second light sources (e.g., light-emitting diodes, or LEDs) that transmit optical radiation at, respectively, red (λ˜630-670 nm) and infrared (λ˜800-1200 nm) wavelengths. The optical module also features a photodetector that detects radiation transmitted or reflected by an underlying artery. Typically the red and infrared LEDs sequentially emit radiation that is partially absorbed by blood flowing in the artery. The photodetector is synchronized with the LEDs to detect transmitted or reflected radiation. In response, the photodetector generates a separate radiation-induced signal for each wavelength. The signal, called a plethysmograph, varies in a time-dependent manner as each heartbeat varies the volume of arterial blood and hence the amount of transmitted or reflected radiation. A microprocessor in the pulse oximeter processes the relative absorption of red and infrared radiation to determine the oxygen saturation in the patient's blood. A number between 94%-100% is considered normal, while a value below 85% typically indicates the patient requires hospitalization. In addition, the microprocessor analyzes time-dependent features in the plethysmograph to determine the patient's heart rate.

Pulse oximeters work best when the appendage they attach to (e.g., a finger) is at rest. If the finger is moving, for example, the light source and photodetector within the optical module typically move relative to the hand. This generates ‘noise’ in the plethysmograph, which in turn can lead to motion-related artifacts in data describing pulse oximetry and heart rate. Ultimately this reduces the accuracy of the measurement. A non-invasive medical device called a sphygmomanometer measures a patient's blood pressure using an inflatable cuff and a sensor (e.g., a stethoscope) that detects blood flow by listening for sounds called the Korotkoff sounds. During a measurement, a medical professional typically places the cuff around the patient's arm and inflates it to a pressure that exceeds the systolic blood pressure. The medical professional then incrementally reduces pressure in the cuff while listening for flowing blood with the stethoscope. The pressure value at which blood first begins to flow past the deflating cuff, indicated by a Korotkoff sound, is the systolic pressure. The stethoscope monitors this pressure by detecting strong, periodic acoustic ‘beats’ or ‘taps’ indicating that the blood is flowing past the cuff (i.e., the systolic pressure barely exceeds the cuff pressure). The minimum pressure in the cuff that restricts blood flow, as detected by the stethoscope, is the diastolic pressure. The stethoscope monitors this pressure by detecting another Korotkoff sound, in this case a ‘leveling off’ or disappearance in the acoustic magnitude of the periodic beats, indicating that the cuff no longer restricts blood flow (i.e., the diastolic pressure barely exceeds the cuff pressure).

Low-cost, automated devices measure blood pressure using an inflatable cuff and an automated acoustic or pressure sensor that measures blood flow. These devices typically feature cuffs fitted to measure blood pressure in a patient's wrist, arm or finger. During a measurement, the cuff automatically inflates and then incrementally deflates while the automated sensor monitors blood flow. A microcontroller in the automated device then calculates blood pressure. Cuff-based blood-pressure measurements such as these typically only determine the systolic and diastolic blood pressures; they do not measure dynamic, time-dependent blood pressure.

Data indicating blood pressure are most accurately measured during a patient's appointment with a medical professional, such as a doctor or a nurse. Once measured, the medical professional manually records these data in either a written or electronic file. Appointments typically take place a few times each year. Unfortunately, in some cases, patients experience ‘white coat syndrome’ where anxiety during the appointment affects the blood pressure that is measured. For example, white coat syndrome can elevate a patient's heart rate and blood pressure; this, in turn, can lead to an inaccurate diagnoses. Various methods have been disclosed for using pulse oximeters to obtain arterial blood pressure values for a patient. One such method is disclosed in U.S. Pat. No. 5,140,990 to Jones et al., for a ‘Method Of Measuring Blood Pressure With a Photoplethysmograph’. The '990 patent discloses using a pulse oximeter with a calibrated auxiliary blood pressure to generate a constant that is specific to a patient's blood pressure. Another method for using a pulse oximeter to measure blood pressure is disclosed in U.S. Pat. No. 6,616,613 to Goodman for a ‘Physiological Signal Monitoring System’. The '613 patent discloses processing a pulse oximetry signal in combination with information from a calibrating device to determine a patient's blood pressure.

### BRIEF SUMMARY OF THE INVENTION

An object of the invention is to provide an inexpensive cuffless monitor that makes an optical measurement from a patient's finger, ear, or other area of the body to determine real-time blood pressure, pulse oximetry, and heart rate. The monitor typically attaches through a wired or wireless connection to a personal computer or cellular telephone, and leverages the processing, display, and power capabilities of these host devices to measure vital signs. During operation the monitor simply collects data from a patient and sends it to the host device for processing and display. In doing this, the monitor contains only a few inexpensive components, such as a small-scale optical system, microcontroller with an analog-to-digital converter, serial-communication electronics, and power-management electronics.

In one aspect, the invention provides a system for measuring blood pressure from a patient that includes: 1) an optical module featuring systems for measuring signals from the patient, serial communication, and power management; 2) an external computing device configured to attach to the optical module, supply power to the optical module, and receive information from the optical module through the system for serial communication; and 3) an algorithm, operating on the external computing device, that processes information received through the system for serial communication to determine the patient's blood pressure.

In another aspect, the invention provides a system for measuring vital signs from a patient that includes: 1) an optical module featuring systems for measuring signals from the patient and serial communication, the optical module configured to interface to an external wireless device to provide information through the system for serial communication; and 2) an algorithm, operating on the external wireless device, that processes information received through the system for serial communication to determine the patient's vital signs.

In embodiments, the system includes an Internet-based system that connects to the external computing or wireless device to supply information, e.g. a calibration table for the patient determined at an earlier time. The optical module typically includes a microprocessor that performs an analog-to-digital conversion, at least one LED, and a photodetector. The microprocessor typically runs a firmware program that digitizes a signal from the photodetector to generate an optical waveform that is then processed with the algorithm running on the external device to determine the patient's blood pressure and other vital signs. The optical module can also include a short-range wireless system, matched to a short-range wireless system within the external device, which transmits information from one device to the other. The short-range wireless system typically operates at least one of the following protocols: Bluetooth, 802.11, 802.15.4.

In another embodiment, the optical module additionally includes an electrode that measures an electrical impulse that is digitized to generate and electrical waveform. In this case, the microprocessor runs a firmware program that analyzes both the optical and electrical waveforms to determine the patient's blood pressure, heart rate, and pulse oximetry.

In yet another embodiment, the optical module is integrated directly into a hand-held wireless device, i.e. on a side or bottom portion of the device. The hand-held wireless device can be a conventional cell phone or wireless personal digital assistant (PDA). With this configuration, a patient carrying the device can measure their vital signs throughout the day.

The invention has many advantages. In particular, the invention quickly and accurately measures vital signs such as blood pressure, heart rate, and pulse oximetry using a simple, low-cost system. Blood pressure measurements are made without using a cuff in a matter of seconds, meaning patients can monitor their vital signs with minimal discomfort. Ultimately this allows patients to measure their vital signs throughout the day (e.g., while at work), thereby generating a complete set of information, rather than just an isolated measurement. Physicians can use this information to diagnose a wide variety of conditions, particularly hypertension and its many related diseases.

The cuffless blood pressure-measuring device of the invention combines all the benefits of conventional blood-pressure measuring devices without any of the obvious drawbacks (e.g., restrictive, uncomfortable cuffs). Its measurement is basically unobtrusive to the patient, and thus alleviates conditions, such as a poorly fitting cuff, that can erroneously affect a blood-pressure measurement.

Once multiple measurements are made, the host device can analyze the time-dependent measurements to generate statistics on a patient's vital signs (e.g., average values, standard deviation, beat-to-beat variations) that are not available with conventional devices that make only isolated measurements. The host device can then send the information through a wireless connection or the Internet to a central computer system, which then displays it on an Internet-accessible website. This way medical professionals can characterize a patient's real-time vital signs during their day-to-day activities, rather than rely on an isolated measurement during a medical check-up. For example, by viewing this information, a physician can delineate between patients exhibiting temporary increases in blood pressure during medical check-ups (i.e. ‘white coat syndrome’) and patients who truly have high blood pressure. With the invention physicians can determine patients who exhibit high blood pressure throughout their day-to-day activities. In response, the physician can prescribe medication and then monitor how this affects the patient's blood pressure.

In general, the current invention measures blood pressure in an accurate, real-time, comprehensive manner that is not possible with conventional blood pressure-monitoring devices.

These and other advantages of the invention will be apparent from the following detailed description and from the claims.

### BRIEF DESCRIPTION OF THE DRAWINGS

FIG. 1A is a semi-schematic view of an optical module for measuring vital signs;

FIG. 1B is a semi-schematic view of a personal computer connected through a USB cable to the optical module of FIG. 1A;

FIG. 1C is a semi-schematic top view of a USB cable connected to the optical module of FIG. 1A;

FIG. 2 is a schematic view of a circuit board within the optical module of FIGS. 1A-C;

FIG. 3A is a semi-schematic view of the optical module and a cuff-based calibration measurement made at a physician's office;

FIG. 3B is a semi-schematic view of the optical module making measurements using a personal computer following the calibration measurement of FIG. 3A;

FIG. 4 is a screen shot generated on an Internet-accessible web site showing information from the optical module of FIG. 1A;

FIG. 5 is a semi-schematic view of an optical module attached to a patient's ear and connected through a short-range wireless connection to a hand-held wireless device;

FIG. 6A is a semi-schematic view of a hand-held wireless device that includes an integrated sensor for measuring vital signs; and

FIG. 6B is a semi-schematic view of the integrated sensor for measuring vital signs of FIG. 6A, including an electrode in addition to an optical module.

### DETAILED DESCRIPTION OF THE INVENTION

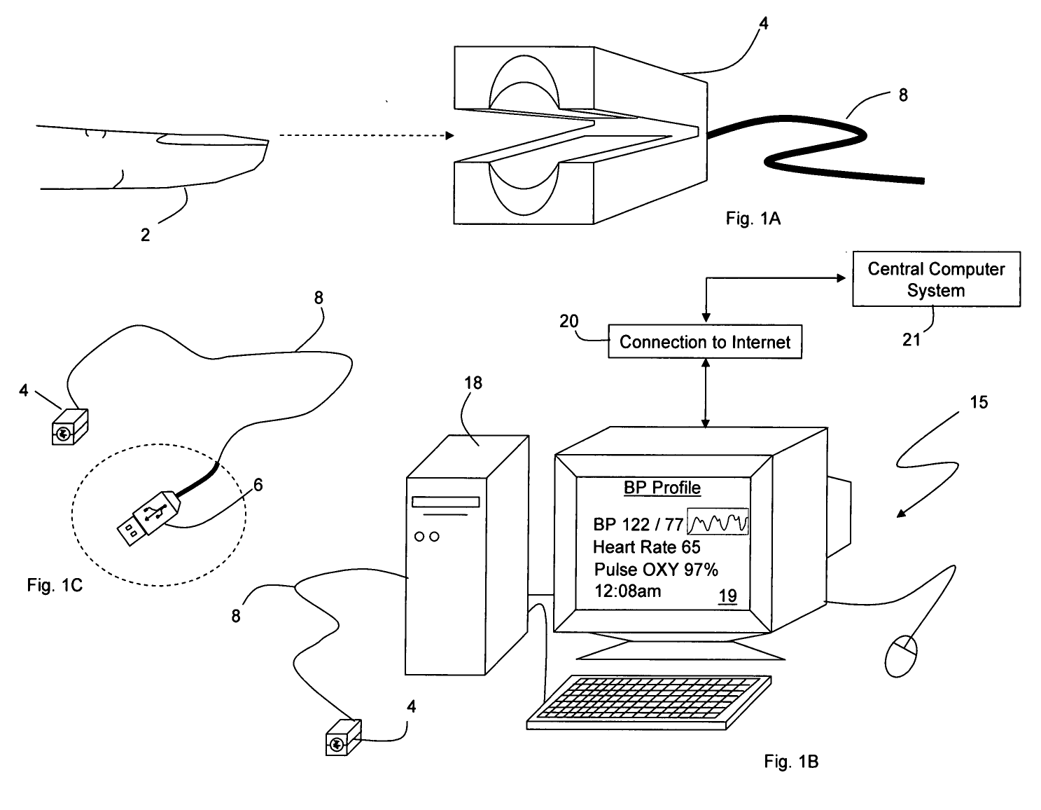

FIGS. 1A-1C show a system 15 for measuring a patient's vital signs that features an inexpensive optical module 4 that clamps to the patient's finger 2 and connects through a cable 8 and USB connector 6 to a personal computer 18. During operation, the optical module 4 measures information describing the patient's vital signs using a small-scale optical system, described below. The module 4 sends this information through the cable 8 and USB connector 6 to the personal computer 18, which processes it and displays properties such as blood pressure, heart rate, and pulse oximetry on the computer's monitor 19. The personal computer 18 also connects to the Internet 20 through which it can download calibration properties and send information to a central computer system 21 for further processing.

The system 15 can be manufactured very inexpensively because it leverages the processing, display, and power capabilities of the personal computer 18. For example, the system uses the microprocessor and memory within the personal computer 18 for processing information from the optical module to determine the patient's vital signs. All information is displayed on the computer's monitor 19 and stored within its internal memory. The optical module 4 is powered through the cable 8 and USB connector 6, meaning that it doesn't need a battery. Information such as calibration properties and vital-sign information are sent and received from the central computer system 21 through the Internet connection 20. Ultimately this means the optical module 4 need only include electronics for measurement, power management, and serial communication. These electronics can be manufactured into a small-scale system for very low cost.

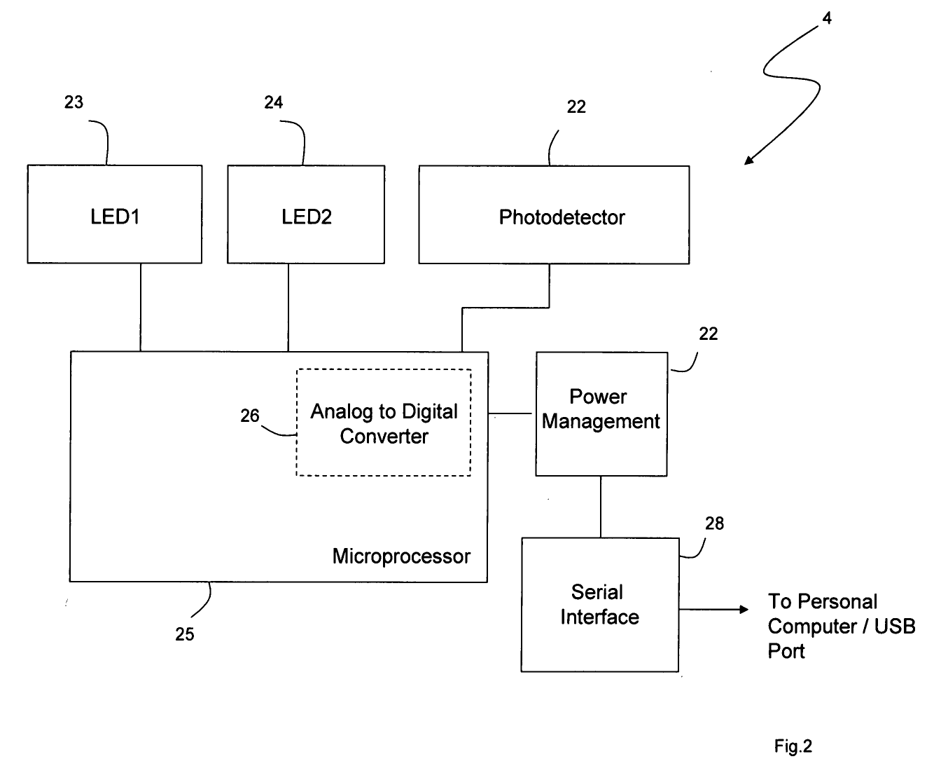

FIG. 2 shows in more detail the electronics within the optical module 4. The module 4 features a pair of LEDs 23, 24 that generate, respectively, red and infrared radiation. A photodetector 22 detects transmitted and scattered radiation and send a radiation-induced photocurrent to an analog-to-digital converter 26 that is embedded into a low-cost microprocessor 25. As the heart pumps blood through the patient's finger, blood cells absorb and transmit varying amounts of the red and infrared radiation depending on how much oxygen binds to the cells' hemoglobin. The photodetector 22 detects transmission at the red and infrared wavelengths, and in response generates a current that the analog-to-digital converter 26 digitizes and converts to a time-dependent optical waveform. The microprocessor 25 receives the optical waveform and sends it through a serial interface 28 to the personal computer from processing. The personal computer analyzes the waveform in combination with calibration parameters as described in detail below to determine the user's vital signs. The analysis used to determine vital signs is described in detail in the pending patent application for a BLOOD PRESSURE MONITORING DEVICE FEATURING A CALIBRATION-BASED ANALYSIS, U.S. patent application Ser. No. 10/967,610, filed Oct. 18, 2004, the contents of which are fully incorporated by reference. The serial interface 28 also connects to a power-management circuit 22 that receives power from the personal computer and processes it to drive the above-described components.

Additional methods for processing vital-sign information measured with the optical module are disclosed in co-pending U.S. patent application Ser. No. 10/810,237, filed Mar. 26, 2004, for a CUFFLESS BLOOD PRESSURE MONITOR AND ACCOMPANYING WEB SERVICES INTERFACE; co-pending U.S. patent application Ser. No. 10/709,015, filed Apr. 7, 2004, for a CUFFLESS BLOOD-PRESSURE MONITOR AND ACCOMPANYING WIRELESS, INTERNET-BASED SYSTEM; or co-pending U.S. patent application Ser. No. 10/752,198, filed Jan. 6, 2004, for a WIRELESS, INTERNET-BASED MEDICAL DIAGNOSTIC SYSTEM, all of which are hereby incorporated by reference in their entirety.

The term ‘microprocessor’, as used herein, preferably means a silicon-based microprocessor or microcontroller that operates compiled computer code to perform mathematical operations on data stored in a memory. Examples include ARM7 or ARM9 microprocessors manufactured by a number of different companies; AVR 8-bit RISC microcontrollers manufactured by Atmel; PIC CPUs manufactured by Microchip Technology Inc.; and high-end microprocessors manufactured by Intel and AMD.

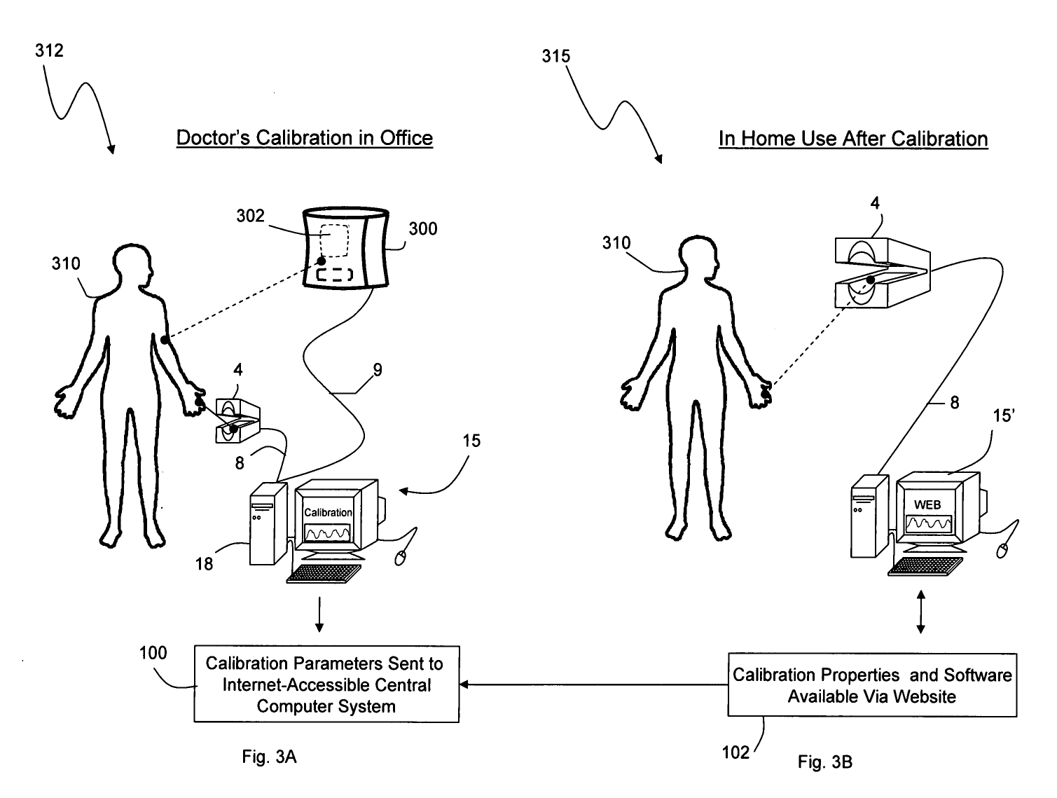

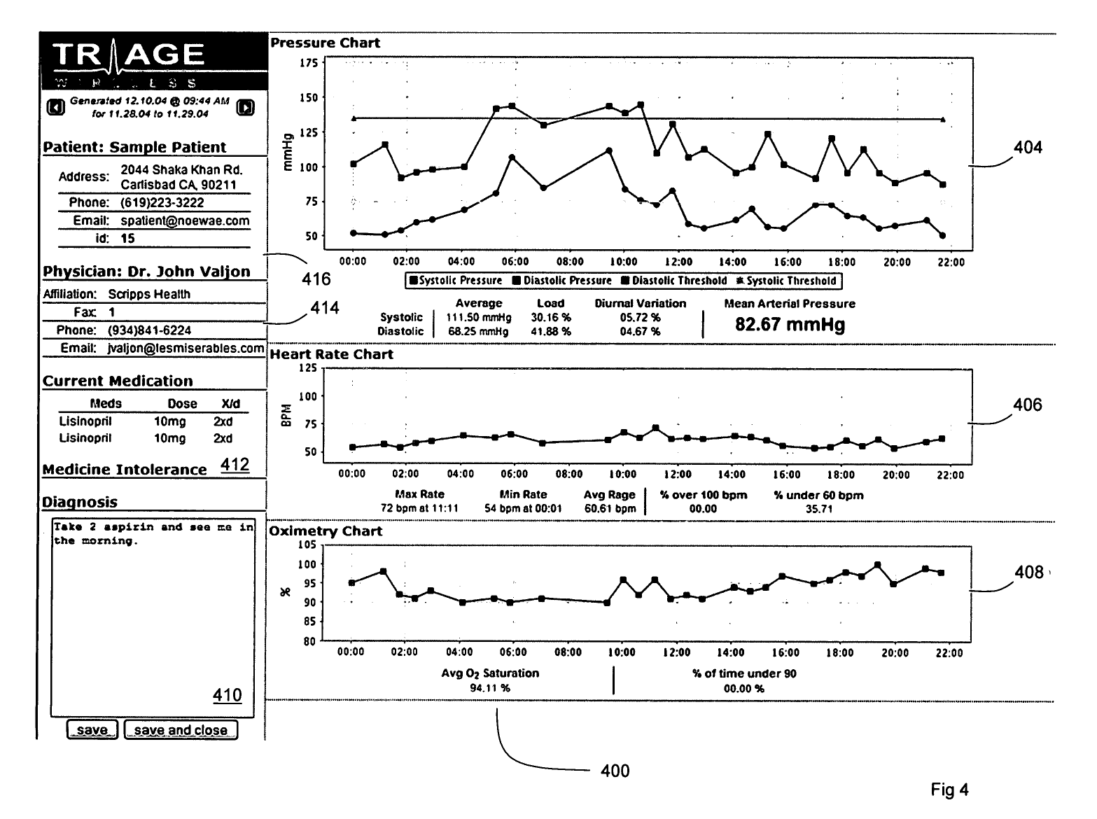

FIGS. 3A, 3B, and 4 show in more detail how the optical module calculates blood pressure from the optical waveform measured with the system shown in FIG. 2. Calibration parameters are preferably determined from a patient 310 in a physician's office using a conventional blood-pressure cuff 300 and the system 15 described with reference to FIGS. 1A-1C. In a preferred embodiment, the blood-pressure cuff 300 temporarily attaches to one of the patient's arms. Immediately prior to measuring the calibration parameters, an electronic system 302 within the blood pressure cuff sends a signal through a cable 9 to the personal computer 18 indicating that the calibration process is about to begin. Once the signal is received, the electronic system 302 and the optical module 4 simultaneously collect, respectively, blood pressure values (systolic, diastolic pressures) and a corresponding optical waveform. The electronic system 302 measures systolic and diastolic blood pressure by controlling a motor-controlled pump and data-processing electronics that generate and analyze Korotokoff sounds as described above. The electronic system 302 sends systolic and diastolic blood pressure values wirelessly to the personal computer through the cable 9 once the calibration measurement is completed. This process is repeated at a later time (e.g., 15 minutes later) to collect a second set of calibration parameters. The blood-pressure cuff 300 is then removed and software running on the computer 18 automatically sends the calibration properties to an Internet-accessible central computer system 100.

The systolic and diastolic blood pressure values measured with the blood-pressure cuff 300, along with their corresponding optical waveforms, are stored in memory in the personal computer 18 and then analyzed with an algorithm to complete the calibration. In one embodiment, for example, the optical waveform is ‘fit’ using a mathematical function that accurately describes its features, and an algorithm (e.g., the Marquardt-Levenberg algorithm) that iteratively varies the parameters of the function until it best matches the optical waveform. This approach is described in detail in the co-pending patent application entitled BLOOD PRESSURE MONITORING DEVICE FEATURING A CALIBRATION-BASED ANALYSIS, the contents of which have been previously incorporated by reference. The mathematical function is typically composed of numerical parameters can be easily stored in memory and analyzed with the personal computer to calibrate the optical module 4.

A number of different properties of the optical waveform correlate to blood pressure, and can thus be analyzed during the calibration process. For example, the optical waveforms typically include primary and reflected ‘pulses’, each corresponding to an individual heartbeat, which can be fit with a number of different mathematical algorithms. Properties of the pulses that correlate to blood pressure include the rate at which they occur (i.e., the heart rate), their width, the time difference between the primary and reflected pulses, the decay time of the pulse, and the amplitude of the both the primary and reflected pulse. Each of these properties can be analyzed during calibration and correlated to blood pressure measured with the calibration device (e.g., the blood-pressure cuff). The personal computer then processes them to generate a calibration table that is stored in memory on the personal computer. After the calibration process, the optical module measures an optical waveform and sends it to the personal computer. The computer processes the waveform with the same process used during calibration to extract the relevant properties. The computer then compares these properties to the calibration table to determine the patient's blood pressure.

Combinations of the calibration parameters may also be used in the blood-pressure measurement. For example, a ratio between the reflected and primary waves' maximum amplitudes may be used as a calibration parameter. In addition, an optical waveform may be numerically processed before it is fit with the mathematical model as a way of maximizing the effectiveness of the fit and consequently the accuracy of the blood-pressure measurement. For example, the personal computer may run an algorithm that takes a second derivative of the waveform as a way of isolating the first and second peaks. This is especially useful if these peaks are merged together within the waveform. In addition, in an effort to improve the signal-to-noise ratio of the optical waveform, the personal computer may average multiple waveforms together. Alternatively, the personal computer reduces high-frequency noise within the optical waveform using a relatively simple multiple-point smoothing algorithm, or a relatively complicated algorithm based on Fourier analysis.

Referring to FIG. 3B, once the calibration is complete the patient 310 leaves the physician's office with the optical module 4 and the USB cable 8, and at a later time plugs this system into their personal computer system 15′ at home or at work. Using a web browser the patient 310 visits a website 102 (e.g., www.triagewireless.com) and downloads a software program from managing the blood pressure measurements, and the calibration parameters determined as described for FIG. 3A. The calibration parameters and the software program are stored on the patient's personal computer 15′ and are used for subsequent measurements. For example, the patient 310 can insert their finger into the optical module 4 at various times during the day. In a matter of seconds the optical module measures and processes the optical waveform as described above to extract the relevant measurement properties. The properties are compared to the calibration tables downloaded from the central computer system to make a blood pressure measurement. This information can then be stored on the personal computer 15′ in a database associated with the software program, and can then be sent to a website where it is viewed by both the physician and the patient at a later time. Or it can be described in a printable report that the patient prints and then brings to the physician during a follow-on medical appointment.

FIG. 4, for example, shows a web-based report 500 generated using the process described above. The report 500 features graphs 404, 406, 408 showing, respectively, how the patient's blood pressure, heart rate, and pulse oximetry vary according to time. Each data point in these graphs represents an individual measurement made with the optical module. The report 500 also includes a section 410 where the patient or physician can record notes on the patient's condition; a section 412 listing the patient's current medication; and sections 414, 416 listing, respectively, the physician's and patient's personal information. Such a report is typically made available on a website that features unique ‘logins’ (e.g., combination of a username and password) for both the physician and patient. The patient's login typically renders a web page that shows only the patient's information, whereas the physician's login renders a web page that includes information for all the patients under the physician's charge.

The same processing capabilities carried out by the personal computer 18 with reference to FIGS. 1A-1C can also be accomplished by a conventional cellular telephone or PDA. These devices typically feature embedded ARM7 or ARM9 microprocessors, along with displays and wired or wireless (e.g., Bluetooth-compatible) serial interfaces, making them well suited to accept and process optical waveforms as described above to determine a patient's vital signs. In particular, mobile devices based on Qualcomm's CDMA technology feature chipsets that integrate both hardware and software for the Bluetooth™ wireless protocol. This means these mobile devices can operate with the above-described blood-pressure monitor with little or no modifications. Such chipsets, for example, include the MSM family of mobile processors (e.g., MSM6025, MSM6050, and the MSM6500). These chipsets are described and compared in detail in http://www.qualcomm.com. For example, the MSM6025 and MSM6050 chipsets operate on both CDMA cellular and CDMA PCS wireless networks, while the MSM6500 operates on these networks and GSM wireless networks. In addition to circuit-switched voice calls, the wireless transmitters used in these chipsets can transmit data in the form of packets at speeds up to 307 kbps in mobile environments.

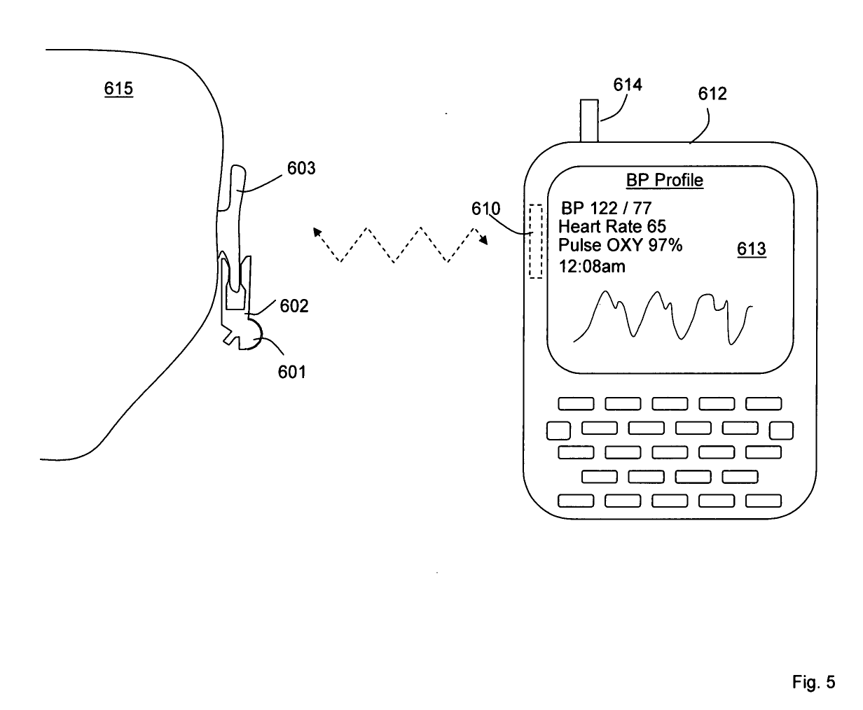

FIG. 5 shows an alternate embodiment of the invention wherein an optical module 602 that attaches to an ear 603 of a patient 615 measures and transmits optical waveforms to a hand-held wireless device 612, e.g. a cellular telephone or a personal digital assistant. The optical module 602 includes a short-range wireless transceiver 601 that sends the waveforms to an embedded, matched short-range wireless transceiver 610 within the hand-held wireless device 612. The optical ear module 602 attaches free from wires to the patient's ear 603 to increase mobility and flexibility. The short-range wireless transceiver 610 preferably operates on a wireless protocol such as Bluetooth™, 802.15.4 or 802.11.

During operation, the optical module 602 is calibrated in a physician's office as described with reference to FIG. 3A, and the calibration table is sent to a central computer system. The central computer system then sends the calibration table and software program to the hand-held wireless device 612. The patient then wears the optical module 602 on their ear, during which it measures optical waveforms and sends them through the short-range wireless transceiver 610 to the matched wireless transceiver 610 in the wireless device 612. The embedded microprocessor in the wireless device 612 receives the waveforms and processes them with the calibration table to determine the patient's vital signs. This information can then be displayed on a display 613 on the wireless device 612. The information can also be wireless transmitted by an antenna 614 through wireless network back to the central computer system, which then renders it on website such as that shown in FIG. 4. A more detailed explanation of how information is sent through a wireless link is found in co-pending patent application for a CUFFLESS BLOOD-PRESSURE MONITOR AND ACCOMPANYING WIRELESS MOBILE DEVICE, U.S. patent application Ser. No. 10/967,511, filed Oct. 18, 2004, the contents of which are fully incorporated herein by reference.

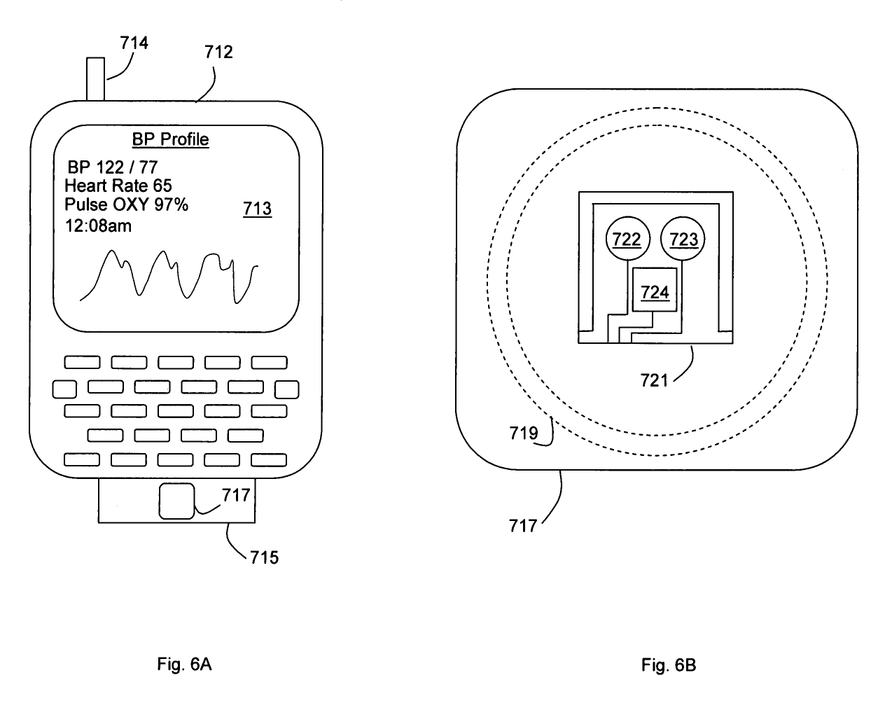

FIG. 6A shows an alternate embodiment of the invention that features a hand-held wireless device 712 that houses an integrated sensor 717 that measures vital signs as described above. In this case, the sensor 717 is embedded directly in a panel 715 that attaches to a bottom portion of the hand-held wireless device 712. During operation, a user places a finger on the sensor 712, which in turn generates information that an algorithm running on a microprocessor within the hand-held wireless device 712 processes to determine the patient's blood pressure and other vital signs. A user interface 713 displays the vital signs directly on the hand-held wireless device 712. Using an antenna 714, the microprocessor can then transmit the vital signs as described above through a wireless network to an Internet-accessible website.

FIG. 6B shows the sensor 717 in more detail. Similar to that described above, the sensor 717 includes a pair of LEDs 722, 723 that generate, respectively, red and infrared radiation. A photodetector 724 detects reflected radiation and sends a radiation-induced photocurrent to an analog-to-digital converter that is embedded within the microprocessor within the hand-held wireless device. As the heart pumps blood through the patient's finger, blood cells absorb and transmit varying amounts of the red and infrared radiation depending on how much oxygen binds to the cells' hemoglobin. The photodetector 724 detects reflected radiation at the red and infrared wavelengths, and in response generates a current that the analog-to-digital converter digitizes and converts to a time-dependent optical waveform. The microprocessor receives the optical waveform and analyzes it in combination with calibration parameters to determine the user's vital signs. The sensor 717 may also include an electrode 719 that detects an electrical impulse from the patient's finger that is used in an algorithm for calculating blood pressure. For example, the electrode 719 may detect an electrical impulse that travels instantaneously from the patient's heart to the finger to generate an electrical waveform. At a later time, a pressure wave propagating through the patient's arteries arrives at the sensor, where the LEDs and photodetector detect it as described above to generate an optical waveform. The propagation time of the electrical impulse is independent of pressure, whereas the propagation time of the pressure wave depends strongly on pressure. An algorithm analyzing the time difference between the arrivals of these signals, i.e. the relative occurrence of the electrical and optical waveforms as measured by the sensor 717, can therefore determine the patient's real-time blood pressure when calibrated with a conventional blood-pressure measurement.

Other embodiments are also within the scope of the invention. For example, optics (i.e., LEDs, photodetector) and associated electronics within the optical module can be embedded in sensors that measure optical waveforms from a variety of locations on a patient's body. For example, the optics can be included in an adhesive patch that is worn on the patient's forehead, head neck, chest, back, forearm, or other locations. In general, any location wherein an optical waveform having can be measured with reasonable signal-to-noise is suitable. In addition, the optical waveforms can be processed with a variety of algorithms to extract the calibration parameters. These algorithms can be based on mathematical operations such as Fourier or Laplace analysis, or other techniques commonly used in signal processing. A variety of mathematical functions can be used while fitting the optical waveforms during calibration and measurement. These include Gaussian, exponential, linear, polynomial, sinusoidal, periodic, impulse, logarithmic, Lorentzian, and other mathematical functions.

In addition, the wireless and Internet-based protocols used to transmit information from the patient to the central computer system can use methodologies other than that described above. For example, information can be sent using Web Services or other XML-based protocols. Wireless networks such as CDMA, GSM, GPRS, Mobitex, Motient, satellite, iDEN are suitable for transmitting information from the patient to the central computer system.

A variety of electrical systems can be used to collect the optical waveforms. Similarly, a variety of software systems can be used to process and display the resultant information. Other vital signs may also be determined with the above-described invention. For example, the optical module can include a semiconductor-based temperature sensor, or may utilize an optical system to measure temperatures from the patient's ear. In another embodiment, the system can take a Fourier transform of the optical waveform to determine the patient's respiratory rate. In still other embodiments, the system may include an ECG system for better characterizing arrhythmias and other cardiac conditions.

The system can also include inputs from other sensors, such as a pedometer (to measure the patient's daily exercise), a scale, or a glucometer. In this embodiment, the pedometer or glucometer may be directly integrated into the hand-held wireless device.

In other embodiments, the hand-held wireless device described above can be replaced with a PDA or laptop computer operating on a wireless network. The wireless device may additionally include a GPS module that receives GPS signals through an antenna from a constellation of GPS satellites and processes these signals to determine a location (e.g., latitude, longitude, and altitude) of the monitor and, presumably, the patient. This location could be used to locate a patient during an emergency, e.g. to dispatch an ambulance. In still other embodiments, patient location information can be obtained using position-location technology (e.g. network-assisted GPS) that is embedded in many wireless devices that can be used for the blood-pressure monitoring system.

In still other embodiments, the wireless device can use a ‘store and forward’ protocol wherein each device stores information when it is out of wireless coverage, and then transmits this information when it roams back into wireless coverage. Still other embodiments are within the scope of the following claims:

1. A system for measuring blood pressure from a patient, comprising:
a blood pressure module comprising both optical and electrical systems for measuring, respectively, optical and electrical signals from the patient, a serial communication system, and a power management system;
an external computing device configured to attach to the blood pressure module, supply power to the blood pressure module, and receive optical and electrical signals from the blood pressure module through the serial communication system; and
an algorithm, operating on the external computing device, that processes the optical and electrical signals received through the serial communication system to determine the patient's blood pressure.

2. The system of claim 1, further comprising an Internet-based system that connects to the external computing device.

3. The system of claim 2, wherein the Internet-based system comprises software configured to supply information for measuring blood pressure to the external computing device.

4. The system of claim 3, wherein the information is a calibration table for the patient determined at an earlier time.

5. A system for measuring blood pressure from a patient, comprising:
a blood pressure module comprising optical and electrical systems for measuring, respectively, optical and electrical signals from the patient, a serial communication system, and a power management system, the blood pressure module configured to attach to an external computing device to receive power and to provide optical and electrical signals through the serial communication system; and
an algorithm, operating on the external computing device, that processes the optical and electrical signals received through the serial communication system to determine the patient's blood pressure.

6. The system of claim 5, further comprising an Internet-based system that connects to the external computing device.

7. The system of claim 6, wherein the Internet-based system comprises software configured to supply information for measuring blood pressure to the external computing device.

8. The system of claim 5, wherein the information is a calibration table for the patient determined at an earlier time.

9. The system of claim 5, wherein the optical module further comprises a microprocessor, at least one LED, and a photodetector.

10. The system of claim 9, wherein the microprocessor comprises a module that performs an analog-to-digital conversion.

11. The system of claim 10, wherein the microprocessor comprises a firmware program that digitizes a signal from the photodetector to generate an optical waveform.

12. The system of claim 11, wherein the external computing device is configured to receive the optical waveform and process it with the algorithm to determine the patient's blood pressure.

13. A system for measuring blood pressure from a patient, comprising:
a blood pressure module comprising optical and electrical systems for measuring, respectively, optical and electrical signals from the patient, and a serial communication system, the blood pressure module configured to interface to an external wireless device to provide the optical and electrical signals through the serial communication system; and
an algorithm, operating on the external wireless device, that processes the optical and electrical signals received through the serial communication system to determine the patient's blood pressure.

14. The system of claim 13, further comprising an Internet-based system that connects to the external wireless device.

15. The system of claim 14, wherein the Internet-based system is configured to supply information for measuring blood pressure to the external wireless device through a wireless interface.

16. The system of claim 15, wherein the information is a calibration table for the patient determined at an earlier time.

17. The system of claim 13, wherein the optical module further comprises a short-range wireless system.

18. The system of claim 17, wherein the short-range wireless system is configured to transmit information describing blood pressure to a matched short-range wireless system within the external wireless device.

19. The system of claim 17, wherein the short-range wireless system operates at least one of the following protocols: Bluetooth, 802.11, 802.15.4.

20. A patch for measuring blood pressure, pulse oximetry, and cardiac arrhythmia values from a patient, comprising:
an optical component comprising at least two light-emitting diodes and a photodetector configured to measure time-resolved optical waveforms generated independently from each light-emitting diode from a region of the patient underneath the optical component;
an electrical component comprising at least one electrode and configured to measure a time-resolved electrical waveform from a region of the patient underneath the electrical component;
a microprocessor configured to receive the time-resolved optical and electrical waveforms and: 1) process the time-resolved optical waveforms generated independently from each light-emitting diode to determine a pulse oximetry value; 2) process the time-resolved electrical waveform generated by the at least one electrode to determine a cardiac arrhythmia value; and 3) process one of the optical waveforms and the electrical waveform to determine a time difference, and then process the time difference to determine a blood pressure value.

21. The patch of claim 20, further comprising a temperature sensor configured to measure a temperature value for the patient.

22. The patch sensor of claim 20, wherein the microprocessor further comprises an algorithm configured to process at least one time-resolved waveform to determine a respiration value for the patient.
